(19) United States
(12) Patent Application Publication
(10) Pub. No.: US 2009/0018189 A1
(43) Pub. Date: Jan. 15, 2009

(54) Hormone Sensitive Lipase Modulators And Methods Of Use

(75) Inventors: Jean-Frederic Sauniere, Aix-en-Provence (FR); Yat S. Or, Watertown, MA (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: DEVIRIS

(21) Appl. No.: 12/146,985

(22) Filed: Jun. 26, 2008

Related U.S. Application Data
(60) Provisional application No. 60/949,325, filed on Jul. 12, 2007.

(51) Int. Cl. A61K 31/216; C07C 229/28; A61P 7/00

(52) U.S. Cl. 514/506; 560/38

(57) ABSTRACT

Disclosed herein are compound useful for inhibition of hormone sensitive lipase, pharmaceutical compositions of these compounds, and methods of treatment using these compounds.

### CROSS-REFERENCE TO RELATED APPLICATIONS

This application claims the benefit of U.S. Provisional Application No. 60/949,325, filed Jul. 12, 2007, the disclosure of which is incorporated by reference in its entirety herein.

### FIELD OF THE INVENTION

The present invention relates to compounds, compositions containing them, and their use for treating medical disorders where it is desirable to modulate the activity of hormone-sensitive lipase.

### BACKGROUND

The overall energy homeostasis of a mammalian system requires a high degree of regulation to ensure the availability of the appropriate substrate at the appropriate time. Plasma glucose levels rise during the post-prandial state, to return to pre-prandial levels within 2-3 hours. During these 2-3 hours, insulin promotes glucose uptake by skeletal muscle and adipose tissue and decreases the release of free fatty acids (FFA) from adipocytes, to ensure that the two substrates do not compete with each other. When plasma glucose levels fall, an elevation in plasma FFA is necessary to switch from glucose to fat utilization by the various tissues.

In individuals with insulin resistance, FFA levels do not fall in response to insulin, as they do in normal individuals, preventing the normal utilization of glucose by skeletal muscle, adipose and liver. Furthermore, there is a negative correlation between insulin sensitivity and plasma FFA levels.

Hormone-sensitive lipase (HSL) is an enzyme, expressed primarily in adipocytes, that catalyses the conversion of triglycerides to glycerol and fatty acids. It is through the regulation of this enzyme that the levels of circulating FFA are modulated. Insulin leads to the inactivation of HSL with a subsequent fall in plasma FFA levels during the post-prandial state, followed by the activation of the enzyme when the insulin concentration falls and catecholamines rise during the post-absorptive period. The activation of HSL leads to an increase in plasma FFA, as they become the main source of energy during fasting.

The activation-inactivation of HSL is primarily mediated through the cAMP-protein kinase A and AMP-dependent kinase pathways. There are compounds like nicotinic acid and its derivatives, which decrease the activation of HSL via these pathways and cause a decrease in lipolysis that leads to a reduction in the FFA levels. These drugs have a beneficial effect in the utilization of glucose and in the normalization of the excess triglyceride synthesis seen in patients with elevated FFA. However, because these pathways are used by other processes in the body, these drugs have severe side effects. Of interest to the present application is the disclosure of co-owned, co-pending U.S. application Ser. No. 11/650,912 and International Application No. PCT/US07/00284, the disclosures of which are each incorporated herein by reference. A need exists for compounds that inhibit HSL.

### SUMMARY

The present invention relates to compounds having a structure of formula (I), pharmaceutical compositions thereof, and to methods of using these compounds and compositions in the treatment, prevention, or amelioration of various diseases and disorders in which hormone-sensitive lipase (HSL) is implicated. Inhibition of HSL can lead to a decrease in plasma free fatty acid (FFA) levels, and the compounds as disclosed herein can be used in treatment of disorders and/or diseases in which decreased levels of plasma FFA is desired, such as insulin resistance, metabolic syndrome X, dyslipidemias, and abnormalities of lipoprotein metabolism.

Thus, one aspect provides a compound of formula (I) and salts, solvates and hydrates, racemates, racemic mixtures and pure enantiomers, diastereomers, homologs, analogs and mixtures thereof,

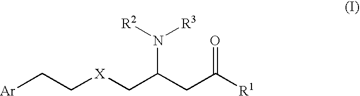

wherein: Ar is an aryl or heteroaryl group; X is —OC(O)—, —NR⁶C(O)—, —(CH₂)ₘ—, —O(CH₂)ₘ, —S(O)(CH₂)ₘ, or —S(O)O(CH₂)ₘ, wherein m is 1 or 2; R¹ is selected from the group consisting of hydrogen, OH, C₁₋₁₀alkyl, aryl, heteroaryl, OC₁₋₁₀alkyl, O-aryl, O-heteroaryl, OC₁₋₁₀alkylenylaryl, OC₁₋₁₀alkylenylheteroaryl, and N(R⁴)R⁵; R², R³, R⁴, R⁵, and R⁶ are each independently selected from the group consisting of hydrogen, C₁₋₁₀alkyl, C(O)C₁₋₁₀alkyl, C(O)C(O)C₁₋₁₀alkyl, C(O)NR⁷R⁸, and C(O)C₁₋₁₀haloalkyl; and R⁷ and R⁸ are independently selected from the group consisting of hydrogen, C₁₋₁₀alkyl, aryl, and heteroaryl.

In some embodiments, R² and R³ are not both hydrogen. In various embodiments, X is OC(O) or NR⁶C(O). In specific embodiments, Ar is phenyl, 4-acetophenyl, 4-hydroxyphenyl, 4-pyridyl, 2-thiophenyl, or 2-furyl. In some specific embodiments, R² is hydrogen, methyl, ethyl, propyl, or benzyl and R³ is C(O)C(O)OCH₃, C(O)CH₃, C(O)CF₃, or C(O)NR⁷R⁸. In various embodiments, R¹ is OH, methoxy, ethoxy, propoxy, phenoxy, benzyloxy, N(CH₃)OCH₃, optionally substituted aryl, or optionally substituted heteroaryl.

In specific embodiments, the compound of formula (I) is as below or a salt, solvate or hydrate, racemate, racemic mixture or pure enantiomer, or mixtures thereof

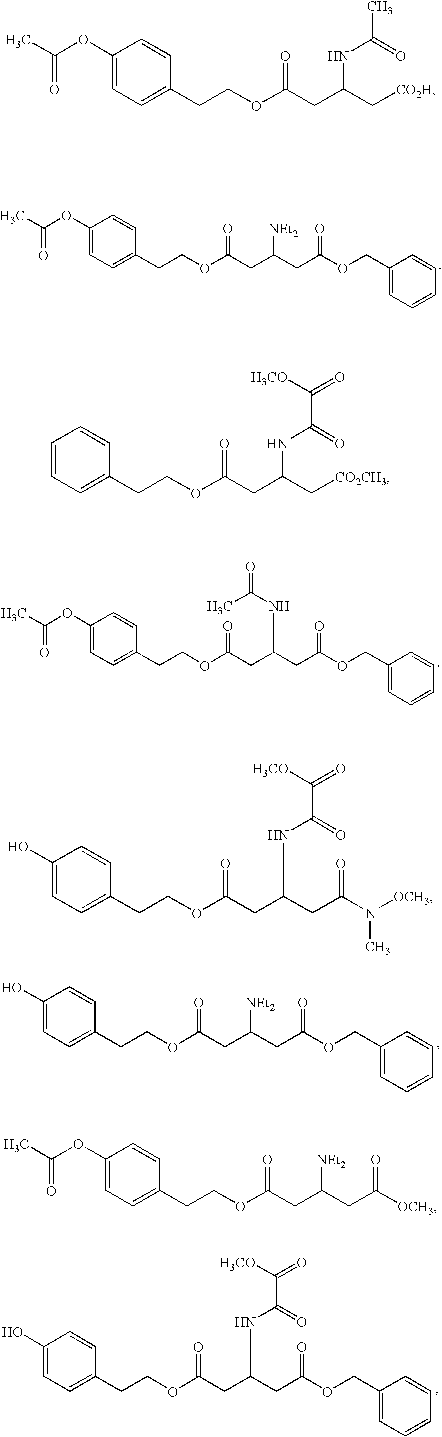

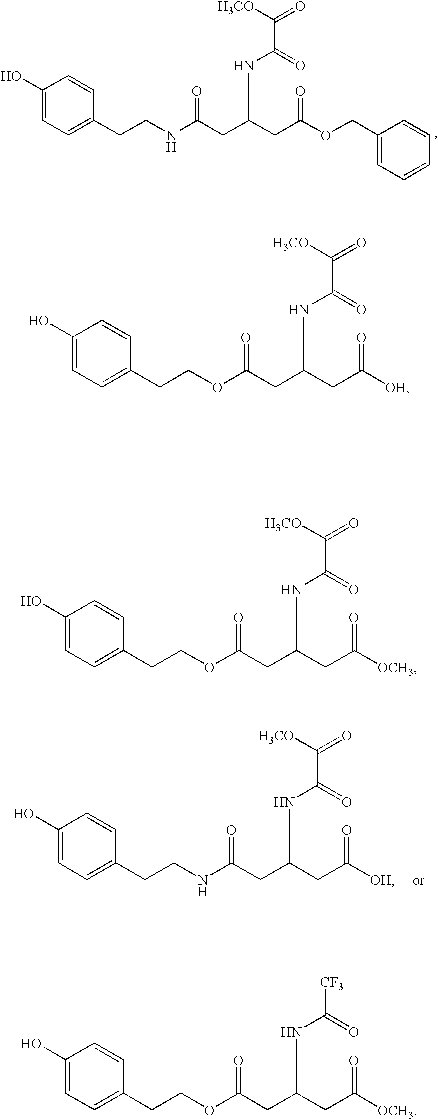

Another aspect provides compositions of the compound of formula (I) as disclosed herein and a pharmaceutically acceptable carrier.

Yet another aspect provides a method of inhibiting hormone sensitive lipase in a patient in need thereof comprising administering a therapeutically effective amount of a compound of formula (I) as disclosed herein which has an IC₅₀ of up to about 10 mM for inhibition of HSL. In some embodiments, the compound of formula (I) has an IC₅₀ of up to about 1 mM or up to about 100 nM. In specific embodiments, the compound is

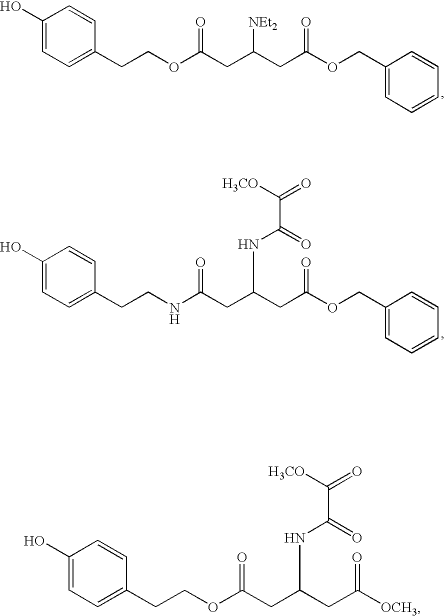

or a salt thereof. In various embodiments, the method further comprises administering a therapeutically effective amount of one or more active agents which has a favorable effect on metabolic disturbances or disorders. In some cases, the active agent and the compound of formula (I) are administered simultaneously. In some embodiments, the method further comprises administering a therapeutically effective amount of one or more anti-diabetic drugs.

Still another aspect of the invention provides a method of treating, preventing, or ameliorating one or more symptoms associated with disorders of fatty acid metabolism or glucose utilization disorders in a patient in need thereof comprising administering to said patient a therapeutically effective amount of a compound as disclosed herein which has an IC₅₀ of up to about 10 mM for inhibition of HSL. In some embodiments, the compound of formula (I) has an IC₅₀ of up to about 1 mM or up to about 100 nM. In specific embodiments, the compound is

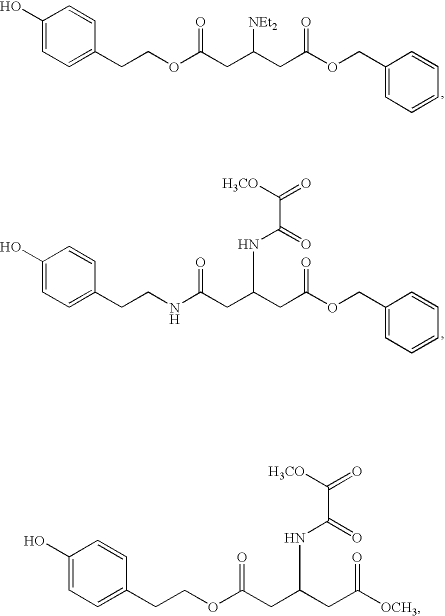

or a salt thereof. In various embodiments, the method further comprises administering a therapeutically effective amount of an active agent for the treatment or prevention of disorders of fatty acid metabolism, glucose utilization disorders, or both. In specific embodiments, the active agent and the compound of formula (I) as disclosed herein are administered simultaneously.

Yet another aspect provides a method of treating, preventing, or ameliorating a disorder involving insulin resistance comprising administering to a patient in need thereof a therapeutically effective amount of a compound of formula (I) as disclosed herein which has an IC₅₀ of up to about 10 mM for inhibition of HSL. In some embodiments, the compound of formula (I) has an IC₅₀ of up to about 1 mM or up to about 100 nM. In specific embodiments,

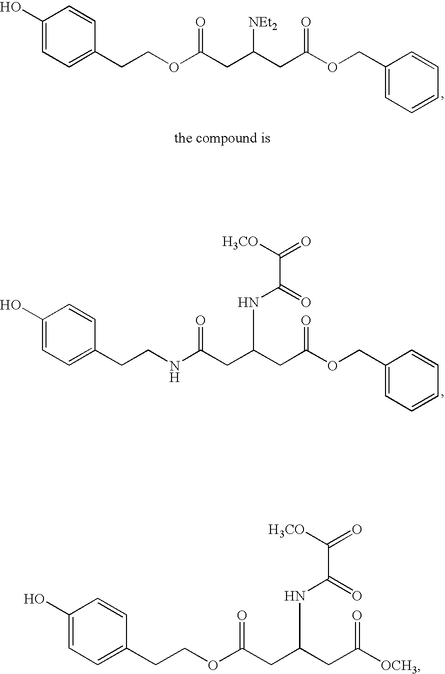

or a salt thereof. In specific cases, the patient suffers from diabetes mellitus. In various embodiments, the method further comprises administering a therapeutically effective amount of an active agent for the treatment, prevention, or amelioration of a disorder in which insulin resistance is involved. In specific embodiments, the active agent and the compound of formula (I) as disclosed herein are administered simultaneously.

Another aspect provides a method of treating, preventing, or ameliorating dyslipidemias or a complication of dyslipidemias comprising administering to a patient in need thereof a therapeutically effective amount of a compound of formula (I) as disclosed herein. In some embodiments, the compound of formula (I) has an IC₅₀ of up to about 1 mM or up to about 100 nM. In specific embodiments, the compound is

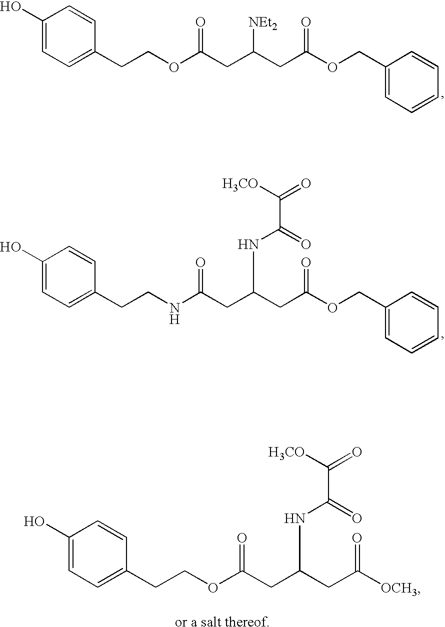

Still another aspect provides a method of treating, preventing, or ameliorating a condition associated with metabolic syndrome X comprising administering to a patient in need thereof a therapeutically effective amount of a compound of formula (I) as disclosed herein. In some embodiments, the compound of formula (I) has an IC₅₀ of up to about 1 mM or up to about 100 nM. In specific embodiments, the compound is

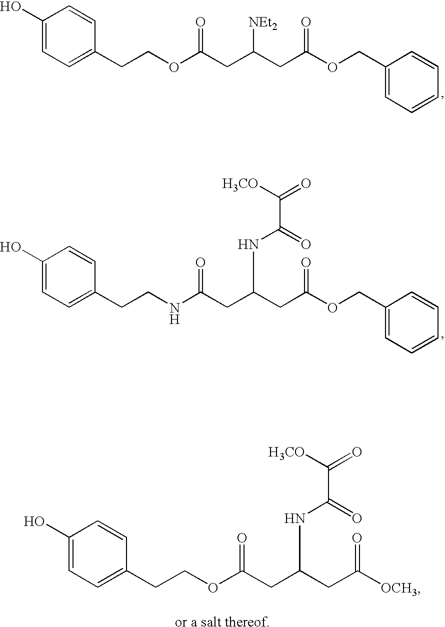

### BRIEF DESCRIPTION OF THE DRAWINGS

FIGS. 1, 2, and 3 show the activity of hormone sensitive lipase (HSL) in the presence of varying concentrations of several compounds as disclosed herein and a reference compound, known to inhibit HSL.

### DETAILED DESCRIPTION

### DEFINITIONS

As used herein, the term “alkyl” refers to straight chained and branched hydrocarbon groups, nonlimiting examples of which include methyl, ethyl, and straight chain and branched propyl and butyl groups. The term “alkyl” includes “bridged alkyl,” i.e., a bicyclic or polycyclic hydrocarbon group, for example, norbomyl, adamantyl, bicyclo[2.2.2]octyl, bicyclo[2.2.1]heptyl, bicyclo[3.2.1]octyl, or decahydronaphthyl. Alkyl groups optionally can be substituted, for example, with hydroxy (OH), halo, aryl, heteroaryl, cycloalkyl, heterocycloalkyl, and amino.

As used herein, the term “alkylene” refers to an alkyl group having a substituent. For example, the term “alkenylenearyl” refers to an alkyl group substituted with a aryl group. The alkylene group is optionally substituted with one or more substituent previously listed as an optional alkyl substituent.

As used herein, the term “aryl” refers to a monocyclic or polycyclic aromatic group, preferably a monocyclic or bicyclic aromatic group, e.g., phenyl or naphthyl. Unless otherwise indicated, an aryl group can be unsubstituted or substituted with one or more, and in particular one to four groups. Exemplary aryl groups include, but are not limited to, phenyl, naphthyl, tetrahydronaphthyl, chlorophenyl, methylphenyl, methoxyphenyl, trifluoromethylphenyl, nitrophenyl, 2,4-methoxychlorophenyl, and the like.

As used herein, the term “heteroaryl” refers to a monocyclic or bicyclic ring system containing one or two aromatic rings and containing at least one nitrogen, oxygen, or sulfur atom in an aromatic ring. Unless otherwise indicated, a heteroaryl group can be unsubstituted or substituted with one or more, and in particular one to four, substituents. Examples of heteroaryl groups include, but are not limited to, thienyl, furyl, pyridyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl.

Suitable aliphatic or aromatic substituents include, but are not limited to, —F, —Cl, —Br, —I, —OH, protected hydroxy, aliphatic ethers, aromatic ethers, oxo, —NO₂, —CN, —C₁-C₁₂-alkyl optionally substituted with halogen (such as perhaloalkyls), C₂-C₁₂alkenyl optionally substituted with halogen, —C₂-C₁₂alkynyl optionally substituted with halogen, —NH₂, protected amino, —NH—C₁-C₁₂alkyl, —NH—C₂-C₁₂alkenyl, —NH—C₂-C₁₂alkynyl, —NH—C₃-C₁₂cycloalkyl, —NHaryl, —NH-heteroaryl, —NH-heterocycloalkyl, -dialkylamino, -diarylamino, -diheteroarylamino, —OC₁-C₁₂-alkyl, —OC₂-C₁₂alkenyl, —OC₂-C₁₂alkynyl, —OC₃-C₁₂cycloalkyl, —Oaryl, —Oheteroaryl, —O-heterocycloalkyl, —C(O)C₁-C₁₂alkyl, —C(O)C₂-C₁₂alkenyl, —C(O)C₂-C₁₂alkynyl, —C(O)C₃-C₁₂cycloalkyl, —C(O)aryl, —C(O)heteroaryl, —C(O)heterocycloalkyl, —CONH₂, —CONHC₁-C₁₂alkyl, —CONHC₂-C₁₂alkenyl, —CONHC₂-C₁₂alkynyl, —CONHC₃-C₁₂cycloalkyl, —CONHaryl, —CONHheteroaryl, —CONHheterocycloalkyl, —CO₂C₁-C₁₂alkyl, —CO₂C₂-C₁₂alkenyl, —CO₂C₂-C₁₂alkynyl, —CO₂C₃-C₁₂cycloalkyl, —CO₂aryl, —CO₂heteroaryl, —CO₂heterocycloalkyl, —OCO₂C₁-C₁₂alkyl, —OCO₂C₂-C₁₂alkenyl, —OCO₂C₂-C₁₂alkynyl, —OCO₂C₃-C₁₂cycloalkyl, —OCO₂aryl, —OCO₂heteroaryl, —OCO₂heterocycloalkyl, —OCONH₂, —OCONHC₁-C₁₂alkyl, —OCONHC₂-C₁₂alkenyl, —OCONHC₂-C₁₂alkynyl, —OCONHC₃-C₁₂cycloalkyl, —OCONHaryl, —OCONHheteroaryl, —OCONHheterocycloalkyl, —NHC(O)C₁-C₁₂alkyl, —NHC(O)C₂-C₁₂alkenyl, —NHC(O)C₂-C₁₂alkynyl, —NHC(O)C₃-C₁₂cycloalkyl, —NHC(O)aryl, —NHC(O)heteroaryl, —NHC(O)heterocycloalkyl, —NHCO₂C₁-C₁₂alkyl, —NHCO₂C₂-C₁₂alkenyl, —NHCO₂C₂-C₁₂alkynyl, —NHCO₂C₃-C₁₂cycloalkyl, —NHCO₂aryl, —NHCO₂heteroaryl, —NHCO₂heterocycloalkyl, —NHC(O)NH₂, NHC(O)NHC₁-C₁₂alkyl, —NHC(O)NHC₂-C₁₂alkenyl, —NHC(O)NHC₂-C₁₂ₐlkynyl, —NHC(O)NHC₃-C₁₂cycloalkyl, —NHC(O)NHaryl, —NHC(O)NHheteroaryl, —NHC(O)NHheterocycloalkyl, NHC(S)NH₂, NHC(S)NHC₁-C₁₂alkyl, —NHC(S)NHC₂-C₁₂alkenyl, —NHC(S)NHC₂-C₁₂alkynyl, —NHC(S)NHC₃-C₁₂cycloalkyl, —NHC(S)NHaryl, —NHC(S)NHheteroaryl, —NHC(S)NHheterocycloalkyl, —NHC(NH)NH₂, —NHC(NH)NHC₁-C₁₂alkyl, —NHC(NH)NHC₂-C₁₂alkenyl, —NHC(NH)NHC₂-C₁₂alkynyl, —NHC(NH)NHC₃-C₁₂cycloalkyl, —NHC(NH)NHaryl, —NHC(NH)NHheteroaryl, —NHC(NH)NHheterocycloalkyl, NHC(NH)C₁-C₁₂alkyl, —NHC(NH)C₂-C₁₂alkenyl, —NHC(NH)C₂-C₁₂alkynyl, —NHC(NH)C₃-C₁₂cycloalkyl, —NHC(NH)aryl, —NHC(NH)heteroaryl, —NHC(NH)heterocycloalkyl, —C(NH)NHC₁-C₁₂alkyl, —C(NH)NHC₂-C₁₂alkenyl, —C(NH)NHC₂-C₁₂alkynyl, —C(NH)NHC₃-C₁₂cycloalkyl, —C(NH)NHaryl, —C(NH)NHheteroaryl, —C(NH)NHheterocycloalkyl, —S(O)C₁-C₁₂alkyl, —S(O)C₂-C₁₂alkenyl, —S(O)C₂-C₁₂alkynyl, —S(O)C₃-C₁₂cycloalkyl, —S(O)aryl, —S(O)heteroaryl, —S(O)heterocycloalkyl, —SO₂NH₂, —SO₂NHC₁-C₁₂alkyl, —SO₂NHC₂-C₁₂alkenyl, —SO₂NHC₂-C₁₂alkynyl, —SO₂NHC₃-C₁₂cycloalkyl, —SO₂NHaryl, —SO₂NHheteroaryl, —SO₂NHheterocycloalkyl, —NHSO₂C₁-C₁₂alkyl, —NHSO₂C₂-C₁₂alkenyl, —NHSO₂C₂-C₁₂alkynyl, —NHSO₂C₃-C₁₂cycloalkyl, —NHSO₂aryl, —NHSO₂heteroaryl, —NHSO₂heterocycloalkyl, —CH₂NH₂, —CH₂SO₂CH₃, -aryl, -arylalkyl, -heteroaryl, -heteroarylalkyl, -heterocycloalkyl, —C₃-C₁₂cycloalkyl, polyalkoxyalkyl, polyalkoxy, -methoxymethoxy, -methoxyethoxy, —SH, —SC₁-C₁₂alkyl, —SC₂-C₁₂alkenyl, —SC₂-C₁₂alkynyl, —SC₃-C₁₂cycloalkyl, —Saryl, —Sheteroaryl, —Sheterocycloalkyl, or methylthiomethyl. It is understood that the aryls, heteroaryls, alkyls and the like can be further substituted.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid or inorganic acid. Examples of pharmaceutically acceptable nontoxic acid addition salts include, but are not limited to, salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid lactobionic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

As used herein, “pharmaceutically acceptable carrier” is intended to include any and all solvents, dispersion media, coatings, antibacterial and antifungal agents, isotonic and absorption delaying agents, and the like, compatible with pharmaceutical administration, such as sterile pyrogen-free water. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

As used herein, the term “therapeutically effective amount” refers to that amount of an active compound that, pursuant to a given mode of administration, results in achieving the desired effect. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The ratio between a toxic and a therapeutically affective does is termed the therapeutic index. Compounds which exhibit high therapeutic indices are preferred. Such data can be used in formulating a range of dosages for use in humans or other animals. The dosage of such compounds preferably lies within a range of circulating concentrations that include the ED₅₀ with little or no toxicity. The dosage can vary within this range depending upon the dosage form employed, and the route of administration utilized.

The compounds disclosed herein can inhibit HSL. Inhibition of HSL is typically reported as IC₅₀, which is a concentration at which 50% of the enzyme is inhibited. The lower the IC₅₀ the more potent the inhibitor. IC₅₀ can be measured using standard techniques. The compounds as disclosed herein can have an IC₅₀ of HSL of up to about 10 mM, up to about 5 mM, up to about 3 mM, up to about 2 mM, up to about 1 mM, up to about 0.5 mM, up to about 0.3 mM, up to about 0.2 mM, up to about 0.1 mM, up to about 50 nM, up to about 40 nM, up to about 30 nM, up to about 25 nM, up to about 20 nM, up to about 15 nM, up to about 10 nM, or up to about 5 nM.

The invention also provides methods for treating, preventing, or ameliorating conditions where a decrease and inhibition of hormone sensitive lipase (HSL) is beneficial to patients to treat disorders where a decreased level of plasma FFA is desired, such as insulin resistance, metabolic syndrome X, dyslipidemia, and abnormalities of lipoprotein metabolism.

The compounds of the invention of the general formula (I) can have a strong inhibitory effect on HSL, an allosteric enzyme in adipocytes which is inhibited by insulin and is responsible for the breakdown of fats in fat cells and thus for transferring fat constituents into the blood stream. Inhibition of this enzyme is therefore equivalent to an insulin-like effect of the compounds and derivatives of the invention, eventually leading to reduction of free fatty acids in the blood and of blood glucose. The compounds of formula (I) can therefore be employed for metabolic derangements such as, for example, for non-insulin-dependent diabetes mellitus, for diabetic syndrome and for direct pancreatic damage.

The compound of formula (I) can be in the form of their salts, solvates and hydrates, racemates, racemic mixtures and pure enantiomers, and to their diastereomers, homologs, analogs and mixtures thereof. In some cases, the compound of formula (I) is a racemic mixture, or an unequal mixture of (R) and (S) enantiomers. In various cases, the compound of formula (I) is predominantly the (S) enantiomer or predominantly the (R) enantiomer. For example,

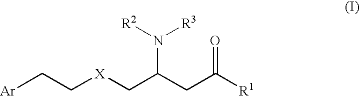

can be predominantly in the form

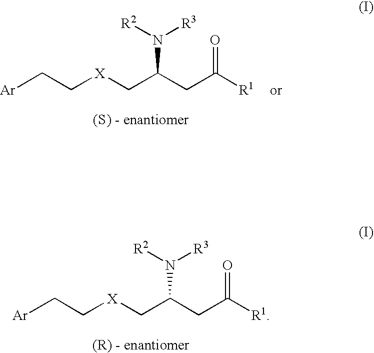

The compounds of the invention of the general formula (I) can have a strong inhibitory effect on HSL, an allosteric enzyme in adipocytes which is inhibited by insulin and is responsible for the breakdown of fats in fat cells and thus for transferring fat constituents into the blood stream. Inhibition of this enzyme is therefore equivalent to an insulin-like effect of the compounds and derivatives of the invention, eventually leading to reduction of free fatty acids in the blood and of blood glucose. The compounds of formula (I) can therefore be employed for metabolic derangements such as, for example, for non-insulin-dependent diabetes mellitus, for diabetic syndrome and for direct pancreatic damage.

### Synthesis of Compounds

The compounds as disclosed herein can be synthesized using the techniques known in the art and those disclosed in International Application No. PCT/US07/00284, filed Jan. 5, 2007, and incorporated by reference herein in its entirety. One synthetic method is outlined below in Scheme 1.

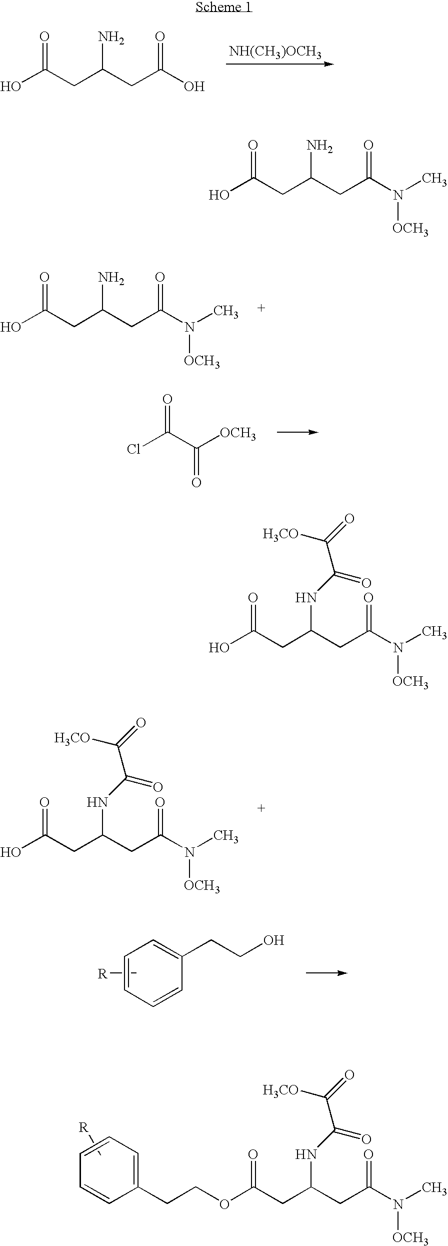

Other compounds of formula (I) can be prepared by altering the starting materials, such as using methyl iodide in place of the methoxymethylamine to provide a methyl ester and/or using an alkylating agent to provide a alkylamine in place of the glycol ester. Other such modifications to the above synthetic scheme and other suitable synthetic means will be apparent to the person of skill in the synthetic arts.

### Therapeutic Uses

The compounds of formula (I) have a strong inhibitory effect on hormone sensitive lipase (HSL), an allosteric enzyme in adipocytes which is inhibited by insulin and is responsible for the breakdown of fats in fat cells and thus for transferring fat constituents into the blood stream. Inhibition of this enzyme is therefore equivalent to an insulin-like effect of the compounds and derivatives of the invention, eventually leading to reduction of free fatty acids in the blood and of blood glucose. These compounds can therefore be employed for metabolic derangements such as, for example, for non-insulin-dependent diabetes mellitus, for diabetic syndrome and for direct pancreatic damage.

Compounds of formula (I) are particularly suitable for the treatment and/or prevention of alterations of fatty acid metabolism and glucose utilization disorders, disorders in which insulin resistance is involved, e.g., diabetes mellitus, especially type-II diabetes, including the prevention of the complications associated therewith. Particular aspects in this connection are hyperglycemia, improvement in insulin resistance, improvement in glucose tolerance, protection of the pancreatic β-cells, and prevention of macro- and microvascular disorders. Compounds of formula (I) are also suitable for the treatment and/or prevention of dyslipidemias their complications such as, for example, atherosclerosis, coronary heart disease, cerebrovascular disorders, etc, especially those (but not restricted thereto) which are characterized by one or more of the following factors: high plasma triglyceride concentrations, high postprandial plasma triglyceride concentrations, low HDL cholesterol concentration, low ApoA lipoprotein concentrations, high LDL cholesterol concentrations, small dense LDL cholesterol particles and high ApoB lipoprotein concentrations.

Various other conditions may be associated with the metabolic syndrome X, such as: obesity, including central obesity, thromboses, heart failure such as, for example (but not restricted thereto), following myocardial infarction, hypertensive heart disease or cardiomyopathy.

In still another aspect, one or more of the compounds of formula (I) are useful for the treatment of hyperglycemia, elevated HbAlc level, hyperinsulinemia, type II diabetes, latent autoimmune diabetes in adults, maturity onset diabetes, beta-cell apoptosis, hemochromatosis induced diabetes, impaired glucose tolerance, impaired fasting glucose, metabolic syndrome X, insulin resistance, impaired lipid tolerance, cystic fibrosis-related diabetes, polycystic ovarian syndrome, and gestational diabetes.

In still another aspect, one or more of the compounds of formula (I) may be useful for the treatment of liver disorders, such as hepatic steatosis and cirrhosis.

In still another aspect, one or more of the compounds of formula (I) may be useful for the treatment of symptoms such as weight loss and cachexia associated with AIDS or an AIDS related diseases. Also, conditions or disorders, such as osteoarthritis; lupus erythematosus (LE) or inflammatory rheumatic disorders such as, for example, rheumatoid arthritis; vasculitis; wasting (cachexia); gout; ischemia/reperfusion syndrome acute respiratory distress syndrome (ARDS); lipodystrophy and lipodystrophic states, also for treating adverse effects of other drugs used to treat various conditions (e.g. following medicaments for treating HIV or tumors).

In still another aspect, one or more of the compounds of formula (I) are useful for the prevention or treatment of obesity, dyslipidemia, diabetic dyslipidemia, hyperlipidemia, hypertriglyceridemia, hyperlipoproteinemia, hypercholesterolemia, hypertension, essential hypertension, acute hypertensive emergency, arteriosclerosis, atherosclerosis, restenosis, intermittent claudication , cardiovascular disease, cardiomyopathy, cardiac hypertrophy, left ventricular hypertrophy, coronary artery disease, early coronary artery disease, heart insufficiency, exercise tolerance, chronic heart failure, mild chronic heart failure, arrhythmia, cardiac dysrythmia, syncopia, heart attack, myocardial infarction, Q-wave myocardial infarction, stroke, acute coronary syndrome, angina pectoris, unstable angina, cardiac bypass reocclusion, diastolic dysfunction, systolic dysfunction, non-Q-wave cardiac necrosis, catabolic changes after surgery, acute pancreatitis, and irritable bowel syndrome.

In still another aspect, one or more of the compounds of formula (I) may be useful for the prevention or treatment of diabetic retinopathy, background retinopathy, preproliferative retinopathy, proliferative retinopathy, macular edema, cataracts, nephropathy, nephrotic syndrome, diabetic nephropathy, microalbuminuria, macroalbuminuria, neuropathy, diabetic neuropathy, polyneuropathy, and diabetic autonomic neuropathy.

In still another aspect, one or more of the compounds of formula (I) may be useful for the prevention or treatment of other disorders or conditions in which inflammatory reactions or cell differentiation may be involved. For example, atherosclerosis such as, for example (but not restricted thereto), coronary sclerosis including angina pectoris or myocardial infarction, stroke vascular restenosis or reocclusion; chronic inflammatory bowel diseases such as, for example, Crohn's disease and ulcerative colitis, pancreatitis; and other inflammatory states.

In still another aspect, one or more of the compounds of formula (I) can be useful for the prevention or treatment of a disease, condition or disorder wherein cholesterol is a precursor. Such diseases, conditions or disorders may relate to testosterone, e.g. male contraception, excessive testosterone levels, and prostate cancer. They may also relate to cortisol or corticotropin, e.g. Cushing disease.

The compounds as disclosed herein can also be useful for the prevention or treatment of cancer. Thus, one or more of the compounds of formula (I) may be useful for the treatment of insulinoma (pancreatic islet cell tumors), e.g. malignant insulinomas and multiple insulinomas, adipose cell carcinomas, e.g. lipocarcinoma adipose cell tumors; pomatous carcinomas such as, for example, liposarcomas; solid tumors and neoplasms such as, for example (but not restricted thereto), carcinomas of the gastrointestinal tract, liver, biliary tract and pancreas; endocrine tumors; carcinomas of the lungs, kidneys, urinary tract, genital tract, and prostate.

The compounds as disclosed herein can also be useful for the prevention or treatment of phaechromocytoma and other diseases with increased catecholamine incretion.

The compounds as disclosed herein can be useful for the prevention or treatment of prostate cancer, e.g. adenocarcinoma, acute and chronic myeloproliferative disorders and lymphomas; angiogenesis, cancer associated cachexia; neurodegenerative disorders, such as Alzheimer's disease, multiple sclerosis, and Parkinson's disease; erythemato-squamous dermatoses such as, psoriasis, acne vulgaris; and other skin disorders and dermatological conditions which are modulated by PPAR, including but not limited to, eczemas and neurodermatitis; dermatitis such as, for example, seborrheic dermatitis or photodermatiti; keratitis and keratoses such as, for example, seborrheic keratoses, senile keratoses, actinic keratosis, photo-induced keratoses or keratosis follicularis, keloids and keloid prophylaxis; warts, including condylomata or condylomata acuminata; human papilloma viral (HPV) infections such as, for example, venereal papillomata, viral warts such as, for example, molluscum contagiosum, leukoplakia, papular dermatoses such as, for example, lichen planus; skin cancer such as, for example, basal-cell carcinomas, melanomas or cutaneous T-cell lymphomas, localized benign epidermal tumors such as, for example, keratoderma, epidermal naevi, high blood pressure, metabolic syndrome X; polycystic ovary syndrome (PCOS); and asthma.

### Combination Therapy

The compounds as disclosed herein can be administered alone or in combination with one or more further pharmacologically active substances which have, for example, favorable effects on metabolic disturbances or disorders frequently associated therewith. Examples of such medicaments are medicaments which lower blood glucose, anti-diabetics, active ingredients for the treatment of dyslipidemias, anti-atherosclerotic medicaments, anti-obesity agents, anti-inflammatory active ingredients, active ingredients for the treatment of malignant tumors, anti-thrombotic active ingredients, active ingredients for the treatment of high blood pressure, active ingredients for the treatment of heart failure and active ingredients for the treatment and/or prevention of complications caused by diabetes or associated with diabetes.

Furthermore, the present compounds may be administered in combination with one or more anti-hypertensive agents. Examples of antihypertensive agents are β-blockers such as alprenolol, atenolol, timolol, pindolol, propranolol and metoprolol, ACE (angiotensin converting enzyme) inhibitors such as benazepril, captopril, alatriopril, enalapril, fosinopril, lisinopril, quinapril and ramipril, calcium channel blockers such as nifedipine, felodipine, nicardipine, isradipine, nimodipine, diltiazem and verapamil, and a-blockers such as doxazosin, urapidil, prazosin and terazosin. Any suitable combination of the compounds according to the invention with one or more of the above-mentioned compounds and optionally one or more further pharmacologically active substances are considered to be within the scope of the present invention.

The one or more further pharmacologically active substances can be combined with one or more of the compounds of formula (I) in particular for a synergistic improvement in the effect. Administration of the active ingredient combination can take place either by separate administration of the active ingredients to the patient or in the form of combination products.

In one embodiment of the invention, one or more of the compounds of formula (I) are administered in combination with an anti-diabetic (see, e.g., Rote Liste 2001, chapter 12 or in the USP Dictionary of USAN and International Drug Names, US Pharmacopeia, Rockville 2001). Antidiabetics include all insulins and insulin derivatives and other fast-acting insulins, GLP-1 receptor modulators.

The orally effective hypoglycemic active ingredients may include but are not limited to, sulfonylureas (such as tolbutamide, glibenclamide, glipizide or glimepiride), biguanides (such as metformin), meglitinides (such as repaglinide), oxadiazolidinediones, thiazolidinediones (such as ciglitazone, pioglitazone, rosiglitazone), glucosidase inhibitors, glucagon antagonists, GLP-1 agonists, DPP-IV inhibitors, potassium channel openers, insulin sensitizers, inhibitors of liver enzymes involved in the stimulation of gluconeogenesis and/or glycogenolysis, modulators of glucose uptake, compounds which alter lipid metabolism and lead to a change in the blood lipid composition, compounds which reduce food intake, PPAR and PXR modulators and active ingredients which act on the ATP-dependent potassium channel of the beta cells.

In one embodiment, one or more of the compounds of formula (I) are administered in combination with a PPARy agonist such as, for example, rosiglitazone or pioglitazone.

In one embodiment, one or more of the compounds of formula (I) are administered in combination with an α-glucosidase inhibitor such as, for example, miglitol or acarbose.

In one embodiment, one or more of the compounds of formula (I) are administered in combination with more than one of the aforementioned compounds, e.g. in combination with a sulfonylurea and metformin, a sulfonylurea and acarbose, repaglinide and metformin, insulin and a sulfonylurea, insulin and metformin, insulin and troglitazone, insulin and lovastatin, etc.

In one embodiment, one or more of the compounds of formula (I) are administered in combination with one or more lipid modulators. Exemplary lipid modulators include, but are not limited to, HMGCoA reductase inhibitor (such as lovastatin, fluvastatin, pravastatin, simvastatin, ivastatin, itavastatin, atorvastatin, rosuvastatin); bile acid reabsorption inhibitors; polymeric bile acid adsorbent (such as, cholestyramine, colesevelam); cholesterol absorption inhibitor (such as ezetimibe, tiqueside, pamaqueside); and an LDL receptor inducer.

In one embodiment, one or more of the compounds of formula (I) are administered in combination with a PPARa agonist.

In one embodiment, one or more of the compounds of formula (I) are administered in combination with a mixed PPAR α/γ agonist such as, for example, AZ 242, and/or Tesaglitazar.

In one embodiment, one or more of the compounds of formula (I) are administered in combination with a fibrate such as, for example, fenofibrate, gemfibrozil, clofibrate, or bezafibrate.

In one embodiment, one or more of the compounds of formula (I) are administered in combination with nicotinic acid or niacin.

In one embodiment, one or more of the compounds of formula (I) are administered in combination with a CETP inhibitor alone such as, e.g. CP-529, 414 (torcetrapib) and in a multiple combination therapy including but not restricted to HMGCoA reductase inhibitor such as lovastatin, fluvastatin , pravastatin, simvastatin, ivastatin, atorvastatin, or rosuvastatin.

In one embodiment, one or more of the compounds of formula (I) are administered in combination with an ACAT inhibitor.

In one embodiment, one or more of the compounds of formula (I) are administered in combination with an MTP inhibitor such as, for example, implitapide.

In one embodiment, one or more of the compounds of formula (I) are administered in combination with an antioxidant.

In one embodiment, one or more of the compounds of formula (I) are administered in combination with a lipoprotein lipase inhibitor.

In one embodiment, one or more of the compounds of formula (I) are administered in combination with an ATP citrate lyase inhibitor.

In one embodiment, one or more of the compounds of formula (I) are administered in combination with a squalene synthetase inhibitor.

In one embodiment, one or more of the compounds of formula (I) are administered in combination with a lipoprotein(s) antagonist.

In another embodiment, one or more of the compounds of formula (I) are administered in combination with an anti-obesity agent. In one embodiment of the invention, the compounds of the formula (I) or (II) are administered in combination with a lipase inhibitor such as, for example, orlistat.

In one embodiment, the further active ingredient or agent is fenfluramine, dexfenfluramin or sibutramine.

In a further embodiment, one or more of the compounds of formula (I) are administered in combination with CART modulators, NPY antagonists, MC4 agonists, orexin antagonists , H3 agonists , TNF agonists, CRF antagonists, CRF BP antagonists , urocortin agonists, β3 agonists , MSH (melanocyte-stimulating hormone) agonists, CCK-A agonists, serotonin reuptake inhibitors (e.g. dexfenfluramine), mixed serotoninergic and noradrenergic compounds , 5HT agonists , bombesin agonists, galanin antagonists, growth hormone (e.g. human growth hormone), growth hormone-releasing compounds , TRH agonists, uncoupling protein 2 or 3 modulators, leptin agonists , DA agonists (bromocriptine, Doprexin), lipase/amylase inhibitors, PPAR modulators, RXR modulators or TR-β agonists.

In one embodiment, the further active ingredient or agent is leptin, dexamphetamine, amphetamine, mazindole or phentermine.

In one embodiment, one or more of the compounds of formula (I) are administered in combination with medicaments having effects on the coronary circulation and the vascular system, such as, for example, ACE inhibitors (e.g. ramipril), medicaments which act on the angiotensin-renine system, calcium antagonists, beta blockers etc.

In one embodiment, one or more of the compounds of formula (I) are administered in combination with medicaments having an anti-inflammatory effect.

In one embodiment, one or more of the compounds of formula (I) are administered in combination with medicaments which are employed for cancer therapy and cancer prevention.

It will be appreciated that every suitable combination of the compounds of the invention with one or more of the aforementioned compounds and optionally one or more other pharmacologically active substances is regarded as falling within the protection conferred by the present invention.

### Pharmaceutical Compositions

The invention encompasses pharmaceutical compositions comprising pharmaceutically acceptable salts of the compounds, or derivatives, analogs, homologs thereof, of the invention as described above. The invention also encompasses pharmaceutical compositions comprising hydrates of the compounds of the invention. The term “hydrate” includes but is not limited to hemihydrate, monohydrate, dehydrate, trihydrate and the like. The invention further encompasses pharmaceutical compositions comprising any solid or liquid physical form of the compound of the invention. For example, the compounds can be in a crystalline form, in amorphous form, and have any particle size. The particles may be micronized, or may be agglomerated, particulate granules, powders, oils, oily suspensions or any other form of solid or liquid physical form.

The compounds of the invention, and derivatives, fragments, analogs, homologs, pharmaceutically acceptable salts or hydrate thereof can be incorporated into pharmaceutical compositions suitable for administration, together with a pharmaceutically acceptable carrier or excipient. Such compositions typically comprise a therapeutically effective amount of any of the compounds above, and a pharmaceutically acceptable carrier. Preferably, the effective amount when treating cancer is an amount effective to selectively induce terminal differentiation of suitable neoplastic cells and less than an amount which causes toxicity in a patient.

Compounds of formula (I) may be administered by any suitable means, including, without limitation, parenteral, intravenous, intramuscular, subcutaneous, implantation, oral, sublingual, buccal, nasal, pulmonary, transdermal, topical, vaginal, rectal, and transmucosal administrations or the like. Pharmaceutical preparations include a solid, semisolid or liquid preparation (tablet, pellet, troche, capsule, suppository, cream, ointment, aerosol, powder, liquid, emulsion, suspension, syrup, injection etc.) containing a compound of formula (I) as an active ingredient, which is suitable for selected mode of administration. In one embodiment, the pharmaceutical compositions are administered orally, and are thus formulated in a form suitable for oral administration, i.e., as a solid or a liquid preparation. Suitable solid oral formulations include tablets, capsules, pills, granules, pellets, sachets and effervescent, powders, and the like. Suitable liquid oral formulations include solutions, suspensions, dispersions, emulsions, oils and the like. In one embodiment, the composition is formulated in a capsule. In accordance with this embodiment, the compositions of the present invention comprise in addition to the active compound and the inert carrier or diluent, a hard gelatin capsule.

Any inert excipient that is commonly used as a carrier or diluent may be used in the formulations of the present invention, such as for example, a gum, a starch, a sugar, a cellulosic material, an acrylate, or mixtures thereof. A preferred diluent is microcrystalline cellulose. The compositions may further comprise a disintegrating agent (e.g., croscarmellose sodium) and a lubricant (e.g., magnesium stearate), and in addition may comprise one or more additives selected from a binder, a buffer, a protease inhibitor, a surfactant, a solubilizing agent, a plasticizer, an emulsifier, a stabilizing agent, a viscosity increasing agent, a sweetener, a film forming agent, or any combination thereof. Furthermore, the compositions of the present invention may be in the form of controlled release or immediate release formulations.

For liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil. Solutions or suspensions can also include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide.

In addition, the compositions may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCI., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol), a glidant (e.g., colloidal silicon dioxide), anti-oxidants (e.g., ascorbic acid, sodium metabisulfite, butylated hydroxyanisole), stabilizers (e.g., hydroxypropyl cellulose, hyroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates) and/or adjuvants.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Patent No. 4,522,811.

It is especially advantageous to formulate oral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

The preparation of pharmaceutical compositions that contain an active component is well understood in the art, for example, by mixing, granulating, or tablet-forming processes. The active therapeutic ingredient is often mixed with excipients that are pharmaceutically acceptable and compatible with the active ingredient. For oral administration, the active agents are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions and the like as detailed above.

Those of ordinary skill in the art would be capable of determining dosing and dosing regimens for the different compounds appropriate for particular disease states and such dosing can be determined empirically. The amount of a compound of the invention necessary to achieve the desired biological effect depends on a number of factors, for example the specific compound chosen, the intended use, the mode of administration and the clinical condition of the patient.

The daily dose is generally in the range from 0.3 mg to 500 mg (typically from 3 mg to 50 mg) per day and per kilogram of bodyweight, for example 3-10 mg/kg/day. An intravenous dose may be, for example, in the range from 0.3 mg to 1.0 mg/kg, which can suitably be administered as slow infusion. Single doses may contain, for example, from 1 mg to 10 g of the active ingredient. Thus, ampoules for injections may contain, for example, from 1 mg to 100 mg, and single-dose formulations which can be administered orally, such as, for example, tablets or capsules, may contain, for example, from 0.05 to 1000 mg, typically from 0.5 to 500 mg.

The daily administration is then repeated continuously for a period of several days to several years. Oral treatment may continue for between one week and the life of the patient. Preferably the administration takes place for five consecutive days after which time the patient can be evaluated to determine if further administration is required. The administration can be continuous or intermittent, i.e., treatment for a number of consecutive days followed by a rest period. The compounds of the present invention may be administered intravenously on the first day of treatment, with oral administration on the second day and all consecutive days thereafter.

The amount of the compound administered to the patient is less than an amount that would cause toxicity in the patient. In the certain embodiments, the amount of the compound that is administered to the patient is less than the amount that causes a concentration of the compound in the patient's plasma to equal or exceed the toxic level of the compounds.

For the therapy of the abovementioned conditions, the compounds of formula (I) may be used as the compound itself, but they are preferably in the form of a pharmaceutical composition with an acceptable carrier. The carrier must, of course, be acceptable in the sense that it is compatible with the other ingredients of the composition and is not harmful for the patient's health. The carrier may be a solid or a liquid or both and is preferably formulated with the compound as a single dose, for example as a tablet, which may contain from 0.05% to 95% by weight of the active ingredient. Other pharmaceutically active substances may likewise be present, including other compounds of the invention. The pharmaceutical compositions of the invention can be produced by one of the known pharmaceutical methods, which essentially consist of mixing the ingredients with pharmacologically acceptable carriers and/or excipients.

Pharmaceutical compositions of the invention are those suitable for oral, rectal, topical, peroral (for example sublingual), intraperitoneal and parenteral (for example subcutaneous, intramuscular, intradermal or intravenous) administration, although the most suitable mode of administration depends in each individual case on the nature and severity of the condition to be treated and on the nature of the compound of formula (I) used in each case. Coated formulations and coated slow-release, PEG, liposomal formulations also belong within the framework of the invention.

Suitable pharmaceutical compounds for oral administration may be in the form of separate units such as, for example, capsules, cachets, chewable tablets or tablets, each of which contain a defined amount of the compound of formula (I); as powders or granules; as solution or suspension in an aqueous or non aqueous liquid; or as an oil-in-water or water-in-oil emulsion. These compositions may, as already mentioned, be prepared by any suitable pharmaceutical method which includes a step in which the active ingredient and the carrier (which may consist of one or more additional ingredients) are brought into contact. The compositions are generally produced by uniform and homogeneous mixing of the active ingredient with a liquid and/or finely divided solid carrier, after which the product is shaped if necessary.

Pharmaceutical compositions which are suitable for peroral (sublingual) administration comprise chewable tablets which contain a compound of formula (I) with a flavoring, normally sucrose and gum arabic, and pastilles which comprise the compound in an inert base such as gelatin and glycerol or sucrose and gum arabic.

Pharmaceutical compositions suitable for parenteral administration comprise preferably sterile aqueous preparations of one or more of the compounds of formula (I), which are preferably isotonic with the blood of the intended recipient. These preparations are preferably administered intravenously, although administration may also take place by subcutaneous, intramuscular or intradermal injection. These preparations can preferably be produced by mixing the compound with water and making the resulting solution sterile and isotonic with blood. Injectable compositions of the invention generally contain from 0.1 to 5% by weight of the active compound.

Pharmaceutical compositions suitable for rectal administration are preferably in the form of single-dose suppositories. These can be produced by mixing one or more of the compounds of formula (I) with one or more conventional solid carriers, for example cocoa butter, and shaping the resulting mixture.

Pharmaceutical compositions suitable for topical use on the skin are preferably in the form of ointment, cream, lotion, paste, spray, aerosol or oil. Carriers which can be used are petrolatum, lanolin, polyethylene glycols, alcohols and combinations of two or more of these substances. The active ingredient is generally present in a concentration of from 0.1 to 15% by weight of the composition, for example from 0.5 to 2%.

Transdermal administration is also possible. Pharmaceutical compositions suitable for transdermal uses. A suitable active ingredient concentration is about 1% to 35%, preferably about 3% to 15%.

The compounds of the formula (I) are distinguished by favorable effects on metabolic disorders. They beneficially influence lipid and glucose metabolism, in particular they lower the triglyceride level and are suitable for the prevention and treatment of type II diabetes and arteriosclerosis and the diverse complications thereof.

The compounds and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

### EXAMPLES

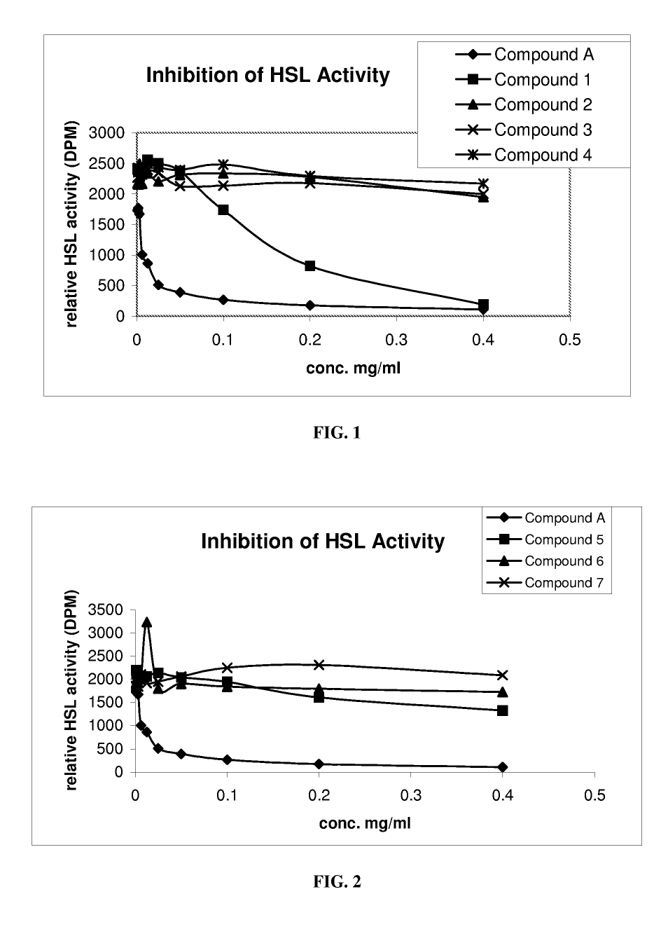

Compounds 1-7 were dissolved in 20% DMSO as 10 mg/mL stock, and used in assays with recombinant HSL to test for the potency for inhibiting HSL activity, the protocol for which is described in International Patent Application No. PCT/US07/00284, which is incorporated by reference in its entirety herein. Each compound's inhibitory effect on HSL is shown in FIG. 1 and 2, with Compound 1 having an IC₅₀ of about 0.1 to about 0.2 mg/mL and Compound 5 showing about 40% inhibition at 0.4 mg/mL.

TABLE 1

| Com- | |
|---|---|
| pound | Structure |
| 1 | 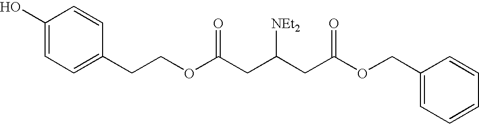 |
| 2 | 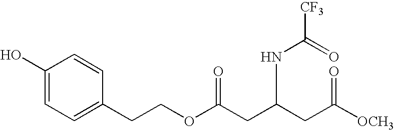 |
| 3 | 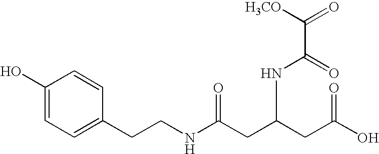 |
| 4 | 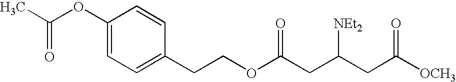 |
| 5 | 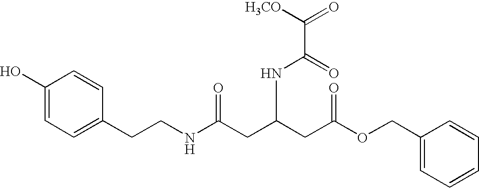 |
| 6 | 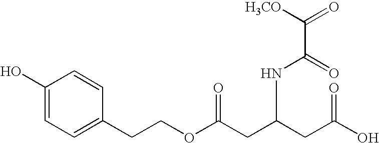 |
| 7 | 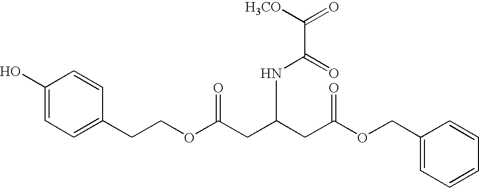 |
| A | 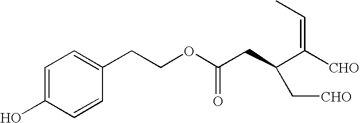 |

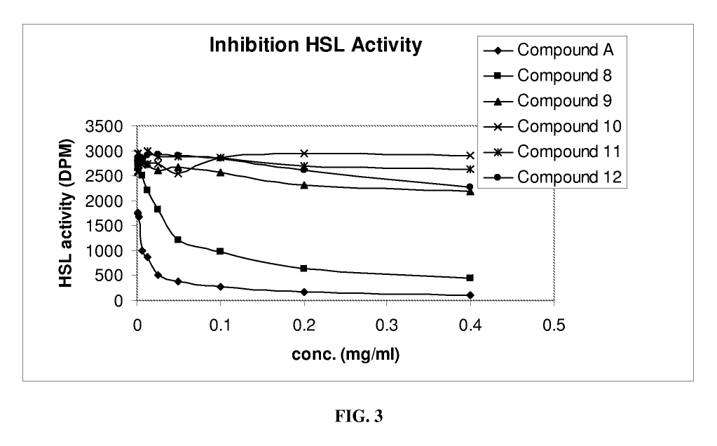

Another set of 5 compounds were tested for inhibitory activity of HSL, Compounds 8-12, the results are shown in Table 2 and FIG. 3, where A is reference compound for HSL inhibitory activity.

TABLE 2

| Com- | |
|---|---|
| pound | Structure |
| 8 | 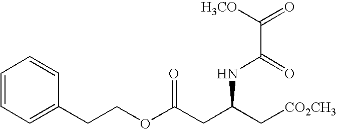 |
| 9 | 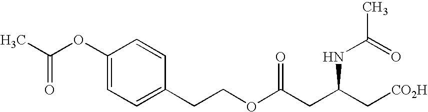 |
| 10 | 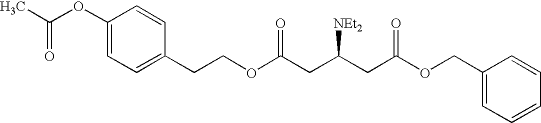 |
| 11 | 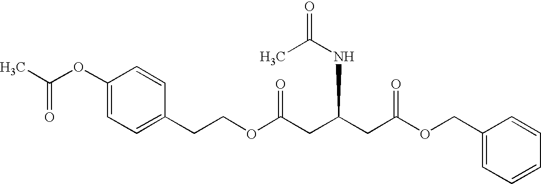 |
| 12 | 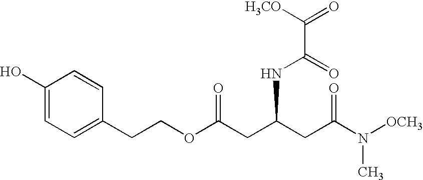 |
| A | 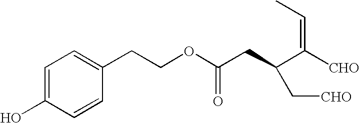 |

All of the above U.S. patents, U.S. patent application publications, U.S. patent applications, foreign patents, foreign patent applications and non patent publications referred to in this specification are incorporated herein by reference, in their entirety.

From the foregoing it will be appreciated that, although specific embodiments of the invention have been described herein for purposes of illustration, various modifications may be made without deviating from the spirit and scope of the invention.

1. A compound of formula (I) 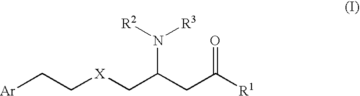
wherein:
Ar is an aryl or heteroaryl group;
X is —OC(O)—, —NR⁶C(O)—, —(CH₂)ₘ—, —O(CH₂)ₘ, —S(O)(CH₂)ₘ, or —S(O)O(CH₂)ₘ, wherein m is 1 or 2;
R¹ is selected from the group consisting of hydrogen, OH, C₁₋₁₀alkyl, aryl, heteroaryl, OC₁₋₁₀alkyl, O-aryl, O-heteroaryl, OC₁₋₁₀alkylenylaryl, OC₁₋₁₀alkylenylheteroaryl, and N(R⁴)R⁵;
R², R³, R⁴, R⁵, and R⁶ are each independently selected from the group consisting of hydrogen, C₁₋₁₀alkyl, C(O)C₁₋₁₀alkyl, C(O)C(O)C₋₁₀alkyl, C(O)NR⁷R⁸, and C(O)C₁₋₁₀haloalkyl; and
R⁷ and R⁸ are independently selected from the group consisting of hydrogen, C₁₋₁₀alkyl, aryl, and heteroaryl,
or a salt thereof.

2. The compound of claim 1, wherein R² and R³ are not both hydrogen.

3. The compound of claim 1, wherein X is OC(O).

4. The compound of claim 1, wherein X is NR⁶C(O).

5. The compound of claim 1, wherein Ar is selected from the group consisting of phenyl, 4-acetophenyl, 4-hydroxyphenyl, 4-pyridyl, 2-thiophenyl, and 2-furyl.

6. The compound of claim 1, wherein R² is selected from the group consisting of hydrogen, methyl, ethyl, propyl, and benzyl and R³ is selected from the group consisting of C(O)C(O)OCH₃, C(O)CH₃, C(O)CF₃, and C(O)NR⁷R⁸.

7. The compound of claim 1, wherein R¹ is selected from the group consisting of OH, methoxy, ethoxy, propoxy, phenoxy, benzyloxy, N(CH₃)OCH₃, optionally substituted aryl, and optionally substituted heteroaryl.

8. The compound of claim 1 selected from the group consisting of

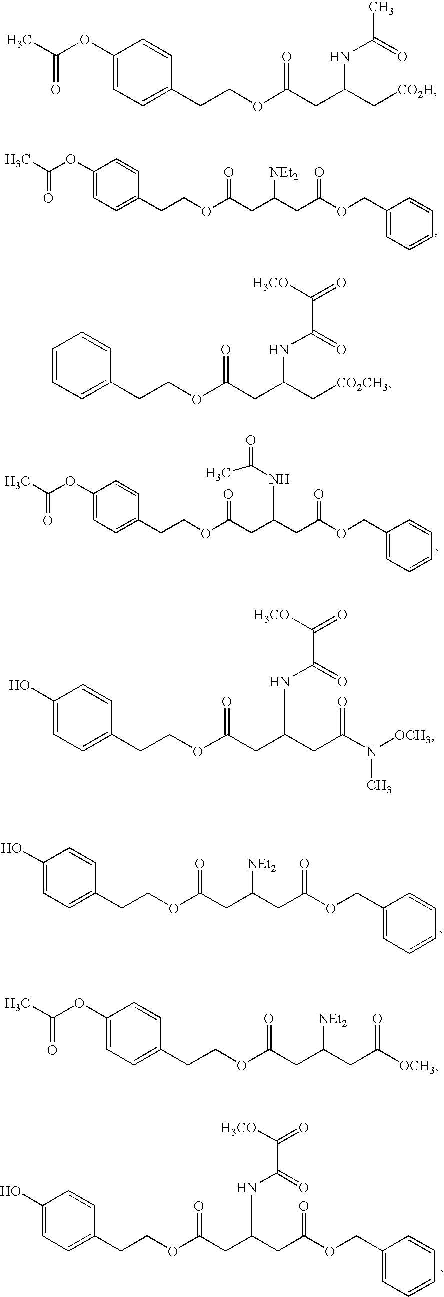

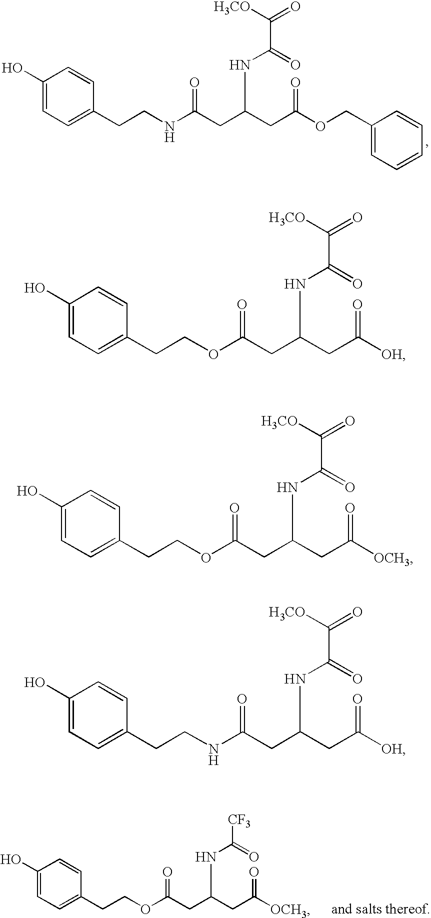

9. A composition comprising a compound of claim 1 and a pharmaceutically acceptable carrier.

10. A method of inhibiting hormone sensitive lipase in a patient in need thereof comprising administering a therapeutically effective amount of a compound of formula (I): 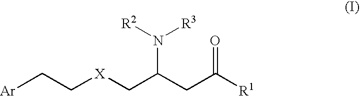
wherein:
Ar is an aryl or heteroaryl group;
X is —OC(O)—, —NR⁶C(O)—, —(CH₂)ₘ—, —O(CH₂)ₘ, —S(O)(CH₂)ₘ, or —S(O)O(CH₂)ₘ, wherein m is 1 or 2;
R¹ is selected from the group consisting of hydrogen, OH, C₁₋₁₀alkyl, aryl, heteroaryl, OC₁₋₁₀alkyl, O-aryl, O-heteroaryl, OC₁₋₁₀alkylenylaryl, OC₁₋₁₀alkylenylheteroaryl, and N(R⁴)R⁵;
R², R³, R⁴, R⁵, and R⁶ are each independently selected from the group consisting of hydrogen, C₁₋₁₀alkyl, C(O)C₁₋₁₀alkyl, C(O)C(O)C₁₋₁₀alkyl, C(O)NR⁷R⁸, and C(O)C₁₋₁₀haloalkyl; and
R⁷ and R⁸ are independently selected from the group consisting of hydrogen, C₁₋₁₀alkyl, aryl, and heteroaryl,
or a salt thereof,
wherein the compound of formula (I) has an IC₅₀ of up to about 10 mM for inhibition of hormone-sensitive lipase (HSL).

11-22. (canceled)

23. A method of treating, preventing, or ameliorating one or more symptoms associated with disorders of fatty acid metabolism or glucose utilization disorders in a patient in need thereof comprising administering to said patient a therapeutically effective amount of a compound of formula (I): 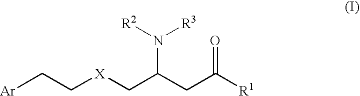
wherein:
Ar is an aryl or heteroaryl group;
X is —OC(O)—, —NR⁶C(O)—, —(CH₂)ₘ—, —O(CH₂)ₘ, —S(O)(CH₂)ₘ, or —S(O)O(CH₂)ₘ, wherein m is 1 or 2;
R¹ is selected from the group consisting of hydrogen, OH, C₁₋₁₀alkyl, aryl, heteroaryl, OC₁₋₁₀alkyl, O-aryl, O-heteroaryl, OC₁₋₁₀alkylenylaryl, OC₁₋₁₀alkylenylheteroaryl, and N(R⁴)R⁵;
R², R³, R⁴, R⁵, and R⁶ are each independently selected from the group consisting of hydrogen, C₁₋₁₀alkyl, C(O)C₁₋₁₀alkyl, C(O)C(O)C₁₋₁₀alkyl, C(O)NR⁷R⁸, and C(O)C₁₋₁₀haloalkyl; and
R⁷ and R⁸ are independently selected from the group consisting of hydrogen, C₁₋₁₀alkyl, aryl, and heteroaryl,
or a salt thereof
wherein the compound of formula (I) has an IC₅₀ of up to about 10 mM for hormone-sensitive lipase (HSL).

24-34. (canceled)

35. A method of treating, preventing, or ameliorating a disorder involving insulin resistance comprising administering to a patient in need thereof a therapeutically effective amount of a compound of claim 1:
wherein the compound of claim 1 has an IC₅₀ of up to about 10 mM for hormone-sensitive lipase (HSL).

36-47. (canceled)

48. A method of treating, preventing, or ameliorating dyslipidemias or a complication of dyslipidemias comprising administering to a patient in need thereof a therapeutically effective amount of a compound of claim 1:
wherein the compound of claim 1 has an IC₅₀ of up to about 10 mM for hormone-sensitive lipase (HSL).

49-57. (canceled)

58. A method of treating, preventing, or ameliorating a condition associated with metabolic syndrome X comprising administering to a patient in need thereof a therapeutically effective amount of a compound of claim 1:
wherein the compound of claim 1 has an IC₅₀ of up to about 10 mM for hormone-sensitive lipase (HSL).

59-67. (canceled)
